(19) United States
(12) Patent Application Publication
(10) Pub. No.: US 2009/0250061 A1
(43) Pub. Date: Oct. 8, 2009

(54) Resuscitation Face Mask

(76) Inventor: Brian L. Marasigan, Houston, TX (US)

Correspondence Address: Klein, O''Neill & Singh, 43 Corporate Park, Suite 204, Irvine, CA, 92606, US

(21) Appl. No.: 12/061,620

(22) Filed: Apr. 2, 2008

(51) Int. Cl. A61M 16/06

(52) U.S. Cl. 128/205.13; 128/205.25

(57) ABSTRACT

An inflatable resuscitation mask for use in hospitals or healthcare clinics for medical purposes including use in anesthesia of a patient. The mask includes several features such as an inflatable/deflatable balloon that allow a perfect seal between the mask and the patient's face in order to prevent gas leakage during oxygenation. The mask also includes a carbon dioxide detecting device that assists the medical operator, physician, or nurse to determine proper ventilation. The mask is designed to fit all facial shapes and sizes of the patients and still provide comfort to the patients during oxygenation.

### BACKGROUND OF THE INVENTION

1. Technical Field

The present invention relates to masks for use in situations requiring the delivery of oxygen, and more specifically, for use on patients with various facial contours and sizes during the administration of anesthesia or resuscitative oxygen.

2. State of the Art

Ventilation masks are employed in numerous life-saving situations to facilitate oxygen delivery to the mouth or nose of the patient. Existing ventilation masks typically comprise of a large cover that fits over the nose and mouth of the patient and may include various features such as valves, tubes, rubber ridges, and balloons. These masks are particularly flexible and made of soft rubber, plastic, or the like.

Ventilation masks can be mouth-to-mouth resuscitation devices or manual and machine ventilation masks. Mouth-to-mouth devices are not often used in medical and surgical facilities. Ventilation or resuscitation masks are generally used for oxygen delivery to patients who do not have a breathing tube in place. These masks are different from plain oxygen masks which are merely used to deliver oxygen to a person who is capable of breathing on his/her own. Ventilation masks are specifically designed to be able to connect machines and devices that deliver oxygen to a person having severe difficulty in breathing or not breathing at all.

The principles of medicine always begin with the airway, breathing, and circulation. The key to cardiopulmonary resuscitation and anesthesiology is control of the airway, breathing, and circulation. Ventilation masks are used during emergencies when patients stop breathing or need assistance in breathing. Usually it is a bridging method to obtaining a secure airway such as an endotracheal breathing tube or laryngeal mask airway that is inserted into the throat.

Mask ventilation is the process of delivering oxygen to a person in need of assistance in breathing. This commonly occurs during respiratory failure. During cardiopulmonary resuscitation (CPR) and operative care, a respiratory therapist, anesthesiologist, or other health caregiver attempts to deliver oxygen to patients. Mask ventilation is important in maintaining oxygen supply to the body while the anesthesiology team prepares to place a breathing tube.

In surgery, there is a period where the patient is subject to anesthesia, and respiration is therefore maintained by an anesthetist. In order for an anesthetist to ventilate the patient properly for respiration, the mask used for oxygen delivery should provide an airtight seal between the mask and the patient's face.

It is, therefore, desirable that these ventilation masks can effectively seal around the face of the patient. Effective sealing requires masks to have the capability to accommodate various facial contours and sizes, facial deformity, or facial features such as beards, large noses, and sunken cheeks. Current masks often come in different sizes and possess some form of flexible seal to fit any variation of facial shapes and sizes. They are usually triangular in shape and fit over the patient's mouth and nose, and are generally pressed against the patient's face to cause an airtight seal. Consequently, patients complain of discomfort or pain from the pressure of the mask.

Existing mask designs fail to consider several important aspects including ergonomics, unusual facial size and shape of the patient, patient's comfort, the mask's connectablity to machines, proper anatomical physiological considerations, facilitation of proper mask ventilation, and detection of proper ventilation.

Ergonomics of masks for operators is still limited among masks available today. A comfortable fit can be very important in an operator's ability to use the mask. In some cases, mask ventilation may be needed for prolonged periods lasting several hours.

One common and obvious attempt to make masks fit a patient's face is to vary the shape of the mask's balloon and to make the balloon larger. This erroneous assumption that the balloon will prevent leaking is not always correct and can sometimes produce discomfort and be difficult for the operator to handle the mask. Furthermore, these masks are often not effective with elder or large patients.

Another common problem in current mask designs is the idea that a flat base and varied shape balloon are adequate. Designs where a common flat base sits above a flexible balloon do not provide balance and cannot effectively stabilize and seal the mask.

Connectablity of the masks to machines has been standardized so that the connector end of the mask fits a standard 22 mm plastic connector piece. Connection is usually done with the connector being perpendicular to the flat base which is not optimal.

The shape and size of a mask are important factors for fit. The mask must consider not only the anatomy of the patient's face but also the patient's comfort during ventilation. A mask that does not fit the patient properly may damage the patient's eyes, hinder the patient's sight, or cause discomfort.

Mask designs must also consider physiologic issues such as dead space. Dead space is the area in the mask where gas is trapped. An excessive dead space will allow mixing of exhaled air with fresh delivered oxygen air, thus, causing the patient to re-breathe carbon dioxide and hinder oxygenation.

The last two considerations, facilitation of proper mask ventilation and detection of proper ventilation, are generally not considered in previous mask designs. These two factors, proper mask technique and detection of gas exchange of oxygen for carbon dioxide in the lungs, are significant to the patient's health.

Ventilation masks are known in the art.

U.S. Pat. No. 3,695,264 entitled “Respiratory Mask,” issued on Oct. 3, 1972 and U.S. Pat. No. 5,429,683 entitled “Face Mask for Breathing,” issued on Jul. 4, 1995 disclose a flexible mask that allows some form of facial fit and sealing during oxygen delivery. However, these devices do not address the problems mentioned above.

None of the references cited above disclose the present invention.

It is the object of the present invention to provide effective mask sealing for all facial contours and sizes of the patients, particularly, for patients subject to anesthesia.

It is also the object of this invention to provide mask stability and comfort.

Yet, another object of this invention is to reduce the amount of dead space and provide a detection of carbon dioxide during gas exchange in order for proper ventilation.

Accordingly, what is needed is an apparatus for overcoming the foregoing deficiencies and ensuring that a sufficient supply of oxygen is optimally delivered to all patients including elder patients and/or patients with unusual facial features.

### SUMMARY OF THE INVENTION

The present invention broadly provides an apparatus to effectively deliver oxygen to patients in need of assistance in breathing. The present apparatus consists of several components including a 1 to 1.5 mm thick hard clear plastic cover, a rubber non-latex balloon, a standard connector with a size of 22 mm Internal Diameter (ID), four prong mask strap hook at the base of the circular 22 mm connector site, and a standard self-sealing inflate-deflate port for adjustment of balloon size.

The present invention allows four distinct embodiments not found in existing models. The first embodiment is a uniquely shaped nose piece with a flat front end, softer rubber composite construction with balloon, and an angled nose fitting bridge. This embodiment avoids injury to patients' eyes, allows proper fit for all facial sizes and shapes of patients, and decreases compression damage to patients' noses and eyes.

The second embodiment is a uniquely shaped connector port angled twenty-five (25°) degrees towards the chin. This embodiment allows for any connected device to angle away from the patients' eyes, thus, preventing claustrophobia and eye damage. This embodiment also promotes proper positioning of the head of the patient in neck extension.

The third embodiment is the unique ergonomic and effective design of the mask dome with rubber grip ridges, an inwardly angled base, a properly sized balloon, a wide width base, wing-like extensions for the chin, and a generally arched shaped base. This embodiment allows for easy and comfortable use of the mask as well as providing stability without obstructing the nose or mouth.

The fourth embodiment is the carbon dioxide (CO2) detector device being integrated within the mask. This embodiment allows for CO2 detection and monitoring ventilation of an awake or unconscious patient during ventilation and allows cycling of colors with each breath.

### BRIEF DESCRIPTION OF THE DRAWINGS

Various features of this apparatus and method, which are believed to be novel, are set forth with particularity in the appended claims. The present apparatus and method may best be understood by reference to the following description, taken in connection with the accompanying drawings:

FIG. 1 is a diagram illustrating the top-connector view of the resuscitation face mask.

FIG. 2 is a diagram illustrating the side view of the resuscitation face mask.

FIG. 3 is a diagram illustrating nose view of the resuscitation face mask.

### DETAILED DESCRIPTION OF THE DRAWINGS

The following detailed description is of the best presently contemplated mode of carrying out the present invention. This description is not to be taken in a limiting sense, but is made merely for the purpose of illustrating general principles of embodiments of the present invention. The scope of the present invention is best defined by the appended claims.

The present invention provides an apparatus for effectively delivering oxygen to a patient in need of assistance in breathing.

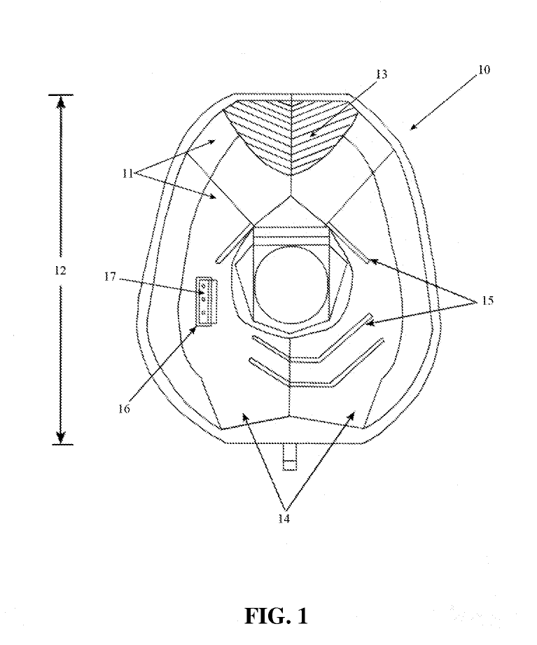

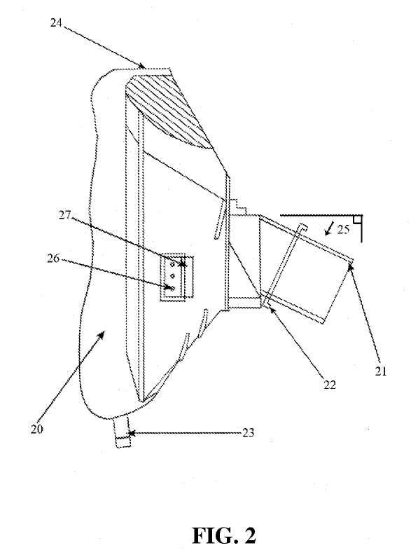

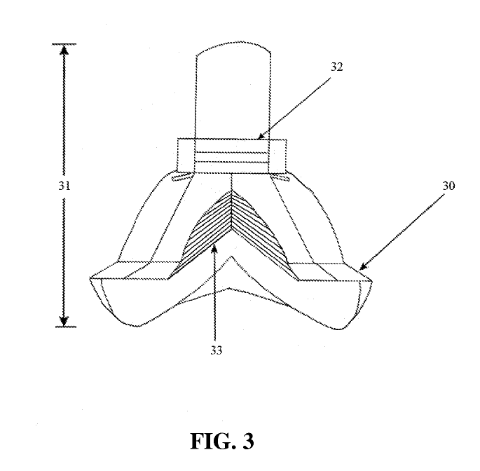

FIGS. 1-3 display the various views of the resuscitation face mask 10. The mask 10 is primarily comprised of a thin, hard, clear plastic 11 with a thickness of 1-1.5 mm molded to design shape. The mask 10 includes a rubber non-latex balloon 20 that makes contact with a patient's face. Certain basic components are included. A connector of 22 mm internal diameter (ID) size 21 is located near the center of the mask 10. The connector 21 has the universal size and shape for airway masks and equipment. The connector 21 attaches the mask to an anesthesia machine or hand ventilation machines such as Mapleson circuit devices. The mask 10 also includes the standard four prong mask strap hook 22 at the base of the circular 22 mm ID connector 21 to be used to hook a universal head strap with four holes to keep the mask in place. The mask 10 has a general length 12 of 110 mm from nose to chin similar to the standard size for a regular adult mask. However, the mask may be made in different sizes to fit different facial structures, i.e. large adult, regular adult, small adult, children of various ages. The mask 10 is designed for both left and right hand use with emphasis for left hand use. The mask 10 also includes a standard self-sealing inflate/deflate port 23 for adjusting the balloon size when necessary.

The shape and angulations of the base 30 are significant in improving control and fit. The balloon 20 has a contoured shape along the entire base to fit the patient's face. The mask 10 has a non-flat base 30. The mask base is anatomically contoured and is inwardly angled to improve seal and stability and decrease balloon size requirement. The height 31 of the mask 10 is lowered to bring the connector base 32 closer to the face and decrease air dead space in the mask 10.

The nose piece 13 of the mask is not pointed and does not press on the patient's eyes as other masks do. The nose piece end is flat 24 and allows for placement on top of the bridge of the nose and below the eyes as opposed to around the bridge of the nose. The nose piece 13 also has a rubber construction and is angled to fit the triangular nose of the patient. The rubber is semi-flexible and non-latex. The arched shape 33 of the nose piece 13 allows for improved fit on all faces including faces with a large nose that often would otherwise prevent mask sealing or cause injury.

The design also includes a slightly angled base 30 for the chin with two wings 14 that cover the sides of the chin in order to improve handling and stability. The mask 10 has rubber ridges 15 designed to improve stability and comfort for the user.

The connector 21 is angled twenty-five degrees 25 toward the chin to prevent risk of injury to the patient's face and avoid blocking the patient's view during mask oxygenation. The twenty-five degree angulation 25 is crucial for proper mask ventilation because the patient's head is often extended upwards to provide a straight and clear passage of air flow from the patient's mouth to his/her lungs. When the patient is lying on a bed in proper ventilation position (with his/her neck extended about twenty-five degrees) and the mask covering his/her face, the connector 21 becomes approximately perpendicular to the bed.

The mask has a small carbon dioxide (CO2) detector 16. Carbon dioxide is only detected if gas exchange is occurring when oxygen is inhaled to the lungs and carbon dioxide is exhaled. CO2 detectors 16 are available in gas analyzing machines used in hospitals and operating rooms. CO2 detection paper can be found in small attachable devices that are attached to the airway circuit to confirm CO2 presence. These devices are solely for CO2 detection and are always sold separately. A pH and CO2 presence is detected by color change on test paper from the chemical reactions of the chemicals and gas. This paper can be inserted inside the mask and is used to detect if there is proper ventilation without the assistance of any other devices merely by showing the appropriate change in color of the paper 17. The paper 17 is visibly located on the side of the mask to avoid being covered by the operator's left hand during oxygenation. There are two common reactions that produce the detected reaction. The paper 17 will change from blue to yellow then yellow to blue based on the pH change of the environment. This reaction is similar to the reaction of litmus paper pH strips. Common known components for the paper 17 include sodium carbonate with thymol blue and glycerol or propylene glycol. Another reaction includes monoethanoloamine with metacrestol purple or thymol blue with propylene glycol. These reactions have been previously documented in the scientific community. The CO2 detector for this device is to be less sensitive in that they require a higher concentration of CO2 to change color. A minimum gas concentration of 1% for any color change would be preferred to the lower <0.6% documented for some detectors. This will prevent false positive readings and allow faster color turnover during mask ventilation.

The CO2 detection component 16 has three ventilation holes 26 that penetrate the entire mask. These holes are small allowing little air to pass to prevent substantial pressure air leak but enough air circulation to renew the CO2 test paper. The inside of the component has an angled air channel 27 that allows air into it from the connector 21 where fresh gas enters the mask.

While the invention herein disclosed has been described by means of specific embodiments and applications thereof, other modifications, variations, and arrangements of the present invention may be made in accordance with the above teachings other than as specifically described to practice the invention within the spirit and scope defined by the following claims.

What is claimed:

1. An inflatable resuscitation face mask capable of delivering gas to a patient, comprising:
a mask body of plastic material;
an inflatable and deflatable balloon;
a circular connector with 22 mm Internal Diameter;
a four prong mask strap hook; and
a carbon dioxide detector.

2. A mask according to claim 1, wherein the plastic mask body has a thickness between 1 to 1.5 mm.

3. The mask according to claim 1, wherein the plastic mask body has a dome shape and covers a patient's face from the nose to the chin of the patient.

4. The mask according to claim 1, wherein the inflatable and deflatable balloon is comprised of a rubber non-latex material.

5. The mask according to claim 1, wherein the inflatable and deflatable balloon is attached to the base of the dome shaped plastic mask body.

6. The mask according to claim 1, wherein the inflatable and deflatable balloon comes in contact with the patient's face.

7. The mask according to claim 1, wherein the inflatable and deflatable balloon has the ability to take the shape of the patient's face so as to form a perfect seal between the mask and the patient's face in order to prevent any gas leakage.

8. The mask according to claim 1, wherein the connector is connected to the center of the mask body.

9. The mask according to claim 1, wherein the connector is angled twenty-five degrees downward towards the chin piece.

10. The mask according to claim 1, wherein the four prong mask strap hook is attached to the base of the circular connector.

11. The mask according to claim 1, wherein the length of the mask body is measured from the patient's nose to the patient's chin.

12. The mask according to claim 1, wherein the mask body can be held by an operator's right or left hand during oxygenation.

13. The mask according to claim 1, wherein the balloon has a self-sealing inflate-deflate port that allows adjustment of the size of said balloon.

14. The mask according to claim 1, wherein said mask has a height between 85 and 95 mm to minimize air dead space in the mask during ventilation.

15. The mask according to claim 1, wherein said mask has a flat-ended nose piece that is placed on the nose and below the eyes of the patient during ventilation.

16. The mask according to claim 1, wherein the nose piece has a flexible non-latex rubber construction that is arched to fit the triangular nose of the patient.

17. The mask according to claim 1, wherein said mask has a slightly angled base with two wings that cover the chin of the patient.

18. The mask according to claim 1, wherein said mask has rubber ridges on its surface to provide stability in the operator's handling of said mask.

19. The mask according to claim 1, wherein said carbon dioxide detector is attached to the inner side of said mask.

20. The mask according to claim 1, wherein said detector has the ability to detect the presence of carbon Dioxide within the mask during ventilation.

21. The mask according to claim 1, wherein said carbon dioxide detector contains a thin strip of paper that changes color to signal to the operator whether there is proper ventilation.
